(19) United States
(12) Patent Application Publication
(10) Pub. No.: US 2022/0142910 A1
(43) Pub. Date: May 12, 2022

(54) COMPOSITION INCLUDING ALL NATURAL BOTANICAL PEPTIDE INGREDIENTS FOR TOPICAL SKIN APPLICATIONS

(71) Applicant: Dianne Martin Bastian, Pelham, NH (US)

(72) Inventor: Dianne Martin Bastian, Pelham, NH (US)

(21) Appl. No.: 17/516,923

(22) Filed: Nov. 2, 2021

Related U.S. Application Data
(60) Provisional application No. 63/110,500, filed on Nov. 6, 2020.
(60) Provisional application No. 63/118,403, filed on Nov. 25, 2020.

(51) Int. Cl. A61K 8/9789; A61K 8/73; A61K 8/64; A61K 8/365; A61K 8/9794; A61K 8/34; A61K 8/67; A61Q 5/00; A61Q 19/08

(52) U.S. Cl. CPC A61K 8/9789; A61K 8/735; A61K 8/645; A61K 8/365; A61Q 19/08; A61K 8/345; A61K 8/673; A61Q 5/002; A61K 8/9794

(57) ABSTRACT

According to an embodiment of the present invention, a formulation for a composition includes active ingredients at an approximate total of 68% by weight of the composition; and filler ingredients at an approximate total of 32% by weight of the composition; wherein the active ingredients include peptides at approximately 30% by weight of the composition; rosewater at approximately 20% by weight of the composition; hyaluronic acid at approximately 15% by weight of the composition; and lactic acid at approximately 3% by weight of the composition; and wherein the filler ingredients include marine plasma; distilled water; and one or more botanicals. The composition is topically applied to areas of the skin. In addition, the formulation may include biotin, and the resulting composition is topically applied to the scalp.

### CROSS-REFERENCE TO RELATED APPLICATIONS

The present application claims the benefit of: (1) U.S. Provisional Patent Application No. 63/110,500, filed Nov. 6, 2020, titled “NON-INJECTABLE COSMETIC DERMAL FILLER FOR TOPICAL APPLICATION”; and (2) U.S. Provisional Patent Application No. 63/118,403, filed Nov. 25, 2020, titled “NON-INVASIVE TOPICAL APPLICATION FOR HAIR RESTORATION”; the contents of both of which are incorporated herein by reference.

### FIELD OF THE INVENTION

The subject matter disclosed herein relates in general to formulations for compositions of topical non-invasive beauty enhancement treatment applications for the skin, and in particular to all natural botanical compositions for specific topical applications such as facial fillers and hair restoration.

### BACKGROUND OF THE INVENTION

Known, prior art all natural botanical peptide compositions are widely used as beauty products for various beauty enhancement treatment applications, including those associated with age management such as facial skin filling and hair restorative growth. Nevertheless, more effective all natural botanical peptide compositions are continually being sought for topical non-invasive beauty enhancement treatment applications for age management purposes.

### BRIEF SUMMARY OF THE INVENTION

An object of embodiments of the present invention is to provide a formulation for a composition of a beauty product that includes all natural botanical peptide ingredients that is effective for beauty enhancement treatment of aging of facial skin and loss of hair on the scalp.

Another object of embodiments of the present invention is to provide a beauty enhancement treatment composition that is easily applied in a non-invasive manner to the skin at specific areas of the body using a portable oxygen machine or an air brush machine similar to one commonly used by an esthetician.

Yet another object of embodiments of the present invention is to provide a composition including all natural botanical peptide ingredients for topical application to the skin at specific areas of the body.

Still another object of embodiments of the present invention is to provide a formulation for a composition for topical application to areas of facial skin that evens out the pigment of the skin and plumps the lips to make them appear fuller.

Another object of embodiments of the present invention is to provide a formulation for a composition that cleans and feeds hair follicles to promote their growth.

Yet another object of embodiments of the present invention is to provide a composition that is relatively easy to topically apply to certain areas of the skin at home using a portable oxygen machine.

Still another object of embodiments of the present invention is to provide a formulation for a composition of all natural botanical peptide ingredients wherein the composition does not include any alcohol, paraffins, or dyes.

According to an embodiment of the present invention, a formulation for a composition includes active ingredients at an approximate total of 68% by weight of the composition; and filler ingredients at an approximate total of 32% by weight of the composition; wherein the active ingredients include peptides at approximately 30% by weight of the composition; rosewater at approximately 20% by weight of the composition; hyaluronic acid at approximately 15% by weight of the composition; and lactic acid at approximately 3% by weight of the composition; and wherein the filler ingredients include marine plasma; distilled water; and one or more botanicals. The composition is topically applied to areas of the skin. In addition, the formulation may include biotin, and the resulting composition is topically applied to the scalp.

### DETAILED DESCRIPTION OF THE INVENTION

It should be understood that, throughout this patent application and specifically in the written description and claims that follow, the terms “about” or “approximately” as used in conjunction with a stated numerical value should be taken to mean extending the range of that numerical value by a percentage value of a minimum of 0.5% on each side of that numerical value and of a maximum of 5.0% on each side of that numerical value, as these ranges would be understood by one of ordinary skill in the relevant art to which embodiments of the present invention pertain.

In exemplary embodiments of the present invention, the composition may be in the form of a liquid solution that is topically applied in a non-invasive manner to the skin at specific locations on the body. However, it should be understood that the broadest scope of the present invention is not limited to a liquid solution of the composition. Instead, various forms of the composition other than liquid (e.g., a solid or semi-solid cream, paste, gel or serum) may be utilized which should be apparent to one of ordinary skill in the art in light of the teachings herein.

Further, it should be understood that a number of the active ingredients and a number of the filler ingredients described in detail hereinafter may each initially be in powder form, which may then be mixed with distilled water or another suitable mixing solution, to form a liquid mixture of the particular ingredient. The liquid ingredient mixtures may then be added to the other ingredients in the formulation as described in more detail hereinafter to form the composition of the overall liquid solution of embodiments of the present invention.

In an exemplary embodiment, a formulation for the composition includes the following ingredients by approximate percentage weight within an overall exemplary amount of the solution: (1) active ingredients at an approximate total of 68% by weight of the overall solution; and (2) filler ingredients at an approximate total of 32% by weight of the overall solution. This exemplary embodiment of the formulation of the composition may be utilized for facial filler applications and, as such, may be applied to the skin at specific locations on the face as described in more detail hereinafter. Also, it should be noted that the each of various active ingredients and the various filler ingredients may be commercially available.

More specifically, the 68% by weight total of the active ingredients may include, for example and without limitation: (1) peptides at approximately 30% by weight; (2) rosewater at approximately 20% by weight; (3) hyaluronic acid at approximately 15% by weight; and (4) lactic acid at approximately 3% by weight. More specifically, the peptides may include, for example and without limitation: (1) pure 6 peptide; (2) palmitoyl oligopeptide; and (3) palmitoyl tetrapeptide-7. Each of these three peptides may be in equal percentages by weight as parts of the 30% by weight of the peptides in the overall solution. In the alternative, each of the weights of the three peptides may differ from each other.

In addition, a relatively small amount of additional peptides may be included in the peptides at approximately 30% by weight within the 68% by weight total of the active ingredients. These additional peptides may include: (1) palmitoyl pentapeptide-3; (2) palmitoyl tripeptide; and (3) acetyl hexapeptide-3. For example, in a 16-ounce amount of liquid solution of the facial filler composition or the hair restoration composition, approximately five drops of a mixture of these additional peptides may be included. The palmitoyl pentapeptide-3 and the palmitoyl tripeptide, together with the palmitoyl oligopeptide, may be commercially available under the brand name Matrixyl®. Also, the acetyl hexapeptide-3 may be commercially available under the brand name Argireline.

The rosewater may include, for example and without limitation, pure hydrosol. Further, the 32% by weight total of the filler ingredients may include, for example and without limitation: (1) marine plasma (sea water); (2) distilled water; and (3) various botanicals, which are also known as “antioxidant extracts” or “extracts.” The marine plasma may comprise, for example and without limitation, purified seawater (Maris sal), aloe vera (Aloe barbadensis), vegetable glycerin, and/or honeysuckle (Lonicera caprifolium).

The distilled water may be used to dilute several of the active ingredients to form mixtures of these active ingredients. For example, each of the pure 6 peptide and the palmitoyl oligopeptide may be mixed with one ounce of distilled water to make one-half teaspoon of the soluble peptide. Also, the palmitoyl tripeptide-7 may be mixed in a ratio of one-quarter teaspoon with one-quarter ounce of distilled water. Similar mixtures of various ones of the active ingredients with distilled water or other suitable mixing solutions should be apparent to one of ordinary skill in the art in light of the teachings herein. These specific mixture amounts described herein are for an exemplary 16-ounce amount of liquid solution of the facial filler composition or the hair restoration composition.

The various botanicals may include, for example and without limitation, any one of, or more than one of in a combination of, the following plant extracts which include: (1) Beta vulagaris (beet) root extract; (2) Aloe barbadensis leaf juice; (3) Cucumis sativus (cucumber) fruit extract; (4) Vitis vinifera (grape) seed extract; (5) Camellia sinensis leaf extract; (6) Althaea officinalis root extract; (7) Anthemis nobilis flower extract; (8) Arnica montana flower extract; (9) hedera helix (ivy) leaf/stem extract; (10) Sambucus nigra flower extract; (11) Hamamelis virginiana (witch hazel) leaf extract; (12) Chamomilla recutita (matricaria) flower oil; and (13) rosa flower oil.

As mentioned hereinabove, each of the various active ingredients and the various filler ingredients may be commercially available and may often be available as “off the shelf” items. For example, certain ones of the peptides and the lactic acid along with certain ones of the filler ingredients may be ingredients in a botanical bio-peptide product, whose specific ingredients may include for example and without limitation: (1) water; (2) glycerin; (3) sodium PCA; (4) benzyl alcohol; (5) polysorbate 20; (6) Beta vulagaris (beet) root extract; (7) hydrolyzed corn starch; (8) butylene glycol; (9) disodium EDTA; (10) panthenol dehydroacetic acid; (11) sodium hydroxide; (12) copper PCA; (13) carbomer; (14) lactic acid; (15) Sloe barbadensis leaf juice; (16) Cucumis sativus (cucumber) fruit extract; (17) Vitis vinifera (grape) seed extract; (18) ascorbic acid; (19) retinyl palmitate; (20) tocopheryl acetate; (21) Camellia sinensis leaf extract; (22) potassium sorbate; (23) sodium benzoate; (24) palmitoyl oligopeptide; and (25) palmitoyl tetrapeptide-7.

In a similar manner, one or more of the various botanicals or plant extracts may be included as ingredients in a commercially available azulene product. More specifically, the ingredients of this azulene product may include for example and without limitation: (1) water (aqua); (2) butylene glycol; (3) methyl gluceth-20; (4) polysorbate 20; (5) Althaea officinalis root extract; (6) Anthemis nobilis flower extract; (7) Arnica montana flower extract; (8) Cucumis sativus (cucumber) fruit extract; (9) hedera helix (ivy) leaf/stem extract; (10) Sambucus nigra flower extract; (11) Hamamelis virginiana (witch hazel) leaf extract; (12) Chamomilla recutita (matricaria) flower oil; (13) rosa flower oil; (14) glycerin; (15) citric acid; (16) sodium citrate; (17) imidazolidinyl urea; and (18) sodium benzoate.

As described hereinbefore, an exemplary formulation for the composition includes: (1) active ingredients at an approximate total of 68% by weight of the overall solution; and (2) filler ingredients at an approximate total of 32% by weight of the overall solution. This exemplary formulation for a 16-ounce amount of the composition may be approximated as follows: (1) 1/16 teaspoon of hyalauronic acid powder; (2) two viles of pure 6 peptide; (3) six ounces of marine plasma; (4) five ounces of peptides; (5) two ounces of azulene; (6) two ounces of rosewater; and (7) 1/32 teaspoon of palmitoyl tripeptide-7 powder.

As described, this exemplary embodiment of the formulation of the composition may be utilized for facial filler applications and, as such, may be applied to the skin at specific locations on the face. In addition, this exemplary embodiment of the formulation of the composition may be utilized for hair restorative applications on the scalp, wherein for these applications the formulation of the composition for facial filler applications may be modified by adding a relatively small amount of biotin (vitamin b7) or its derivative known as biotinol. For example, in a 16-ounce amount of liquid solution of the facial filler composition, approximately five drops of the biotin may be utilized for hair restoration applications.

Once a desired amount of the composition has been prepared by mixing together the various ingredients as per the exemplary embodiments of the formulation described hereinabove, the resulting liquid solution may be applied in a non-invasive manner to selected areas of the skin or scalp. In an exemplary embodiment, a portable oxygen machine or an air brush machine may be utilized to topically apply the liquid solution to freshly washed areas of the skin or scalp. A suitable cleanser (e.g., an enzyme cleanser) may be used to clean the selected areas of the skin or scalp prior to topical application of the solution.

A portable oxygen machine similar to an esthetician's air brush makeup machine may be used to deliver or apply the solution to the skin in a non-injectable or non-invasive manner. Note that the portable oxygen machine is not used to deliver oxygen for breathing or for any other medical purposes. Indeed, the present invention does not relate in any way to a type of treatment of medical conditions. Instead, the present invention relates strictly to a beauty product and the delivery system or machine utilized is solely for the non-invasive delivery of the beauty product to the skin. Also, any type of delivery system or device may be utilized that is suitable for application of beauty enhancement treatments by topically applying a liquid solution to specific areas of the skin or scalp. The portable oxygen machine or air brush machine typically delivers pure air that is pumped out at a relatively high rate to allow the accompanying liquid solution to penetrate and fill the pores of the skin or the follicles of the hair to act in a similar manner as an injectable filler would.

Specifically, the gun of the air brush machine may be held above and close to the area of the skin or scalp to be treated (e.g., two inches from the skin). Next, for skin applications, the spraying of the solution can begin at the center of the face, chin, or neck and outwards or upwards towards the edges (e.g., forehead). This can be repeated two or three times. A greater amount of spraying time can be directed to certain areas of concern on the face such as deep lines and wrinkles, sunken undereye areas, hollows, loose skin, etc. The solution fills in and plumps the skin including at the lips, while also smoothing out the eye areas and evening out the pigment or melanin of the skin. The quality and quantity of the results achieved depend on the specific nature of the skin and how often the solution is applied.

For scalp applications, the spraying of the solution can be carried out back and forth across area of scalp to be treated. A relatively greater amount of the solution can be sprayed than used on the face. A greater amount of spraying time can be directed to certain areas of concern on the scalp such as where the hair is thinner, areas affected by alopecia, etc.

After the solution has been completely applied to the facial areas or to the scalp area, the solution should be allowed to absorb into the skin or scalp without use of hands for an amount of time. A cream may then be used on the face. The solution of embodiments of the present invention can be applied twice a day (once in the morning and once in the evening), or more often as needed. After use, the air brush will likely need to be taken apart and cleaned to prevent any clogging of the needle which is typically part of the air brush gun.

The terminology used herein is for the purpose of describing particular embodiments of the invention only and is not intended to be limiting of the invention. As used herein, the singular forms “a,” “an,” and “the” are intended to include the plural forms as well, unless the context clearly indicates otherwise. It will be further understood that the terms “comprises” and/or “comprising,” when used in this specification, specify the presence of stated features, integers, steps, operations, elements, and/or components, but do not preclude the presence or addition of one or more other features, integers, steps, operations, element components, and/or groups thereof.

While the invention is provided in detail in connection with only a limited number of embodiments, it should be readily understood that the invention is not limited to such disclosed embodiments. Rather, the invention can be modified to incorporate any number of variations, alterations, substitutions, or equivalent arrangements not heretofore described, but which are commensurate with the spirit and scope of the invention. Additionally, while various embodiments of the invention have been described, it is to be understood that the exemplary embodiments may include only some of the described exemplary aspects. Accordingly, the invention it not to be seen as limited by the foregoing description but is only limited by the scope of the appended claims.

1. A formulation for a composition for topical application, comprising:
(a) active ingredients at an approximate total of 68% by weight of the composition; and
(b) filler ingredients at an approximate total of 32% by weight of the composition.

2. The formulation of claim 1, wherein the active ingredients comprise:
(a) peptides at approximately 30% by weight of the composition;
(b) rosewater at approximately 20% by weight of the composition;
(c) hyaluronic acid at approximately 15% by weight of the composition; and
(d) lactic acid at approximately 3% by weight of the composition.

3. The formulation of claim 2, wherein the peptides comprise:
(a) pure 6 peptide;
(b) palmitoyl oligopeptide; and
(c) palmitoyl tetrapeptide-7.

4. The formulation of claim 3, wherein each of the peptides is in an equal amount in the formulation.

5. The formulation of claim 2, wherein the active ingredients further comprise:
(a) palmitoyl pentapeptide-3;
(b) palmitoyl tripeptide; and
(c) acetyl hexapeptide-3.

6. The formulation of claim 2, wherein the rosewater comprises pure hydrosol. The formulation of claim 1, wherein the filler ingredients comprise:
(a) marine plasma;
(b) distilled water; and
(c) one or more botanicals.

8. The formulation of claim 7, wherein the marine plasma comprises:
(a) purified seawater;
(b) aloe vera;
(c) vegetable glycerin; and
(d) honeysuckle.

9. The formulation of claim 7, wherein the one or more botanicals are from the group comprising:
(a) Beta vulagaris (beet) root extract;
(b) Aloe barbadensis leaf juice;
(c) Cucumis sativus (cucumber) fruit extract;
(d) Vitis vinifera (grape) seed extract;
(e) Camellia sinensis leaf extract;
(f) Althaea officinalis root extract;
(g) Anthemis nobilis flower extract;
(h) Arnica montana flower extract;
(i) Hedera helix (ivy) leaf/stem extract;
(j) Sambucus nigra flower extract;
(k) Hamamelis virginiana (witch hazel) leaf extract;
(l) Chamomilla recutita (matricaria) flower oil; and
(m) rosa flower oil.

10. The formulation of claim 7, wherein the distilled water mixes with one or more of the active ingredients to form a dilution mixture thereof.

11. The formulation of claim 1, wherein the active ingredients further comprise biotin.

12. The formulation of claim 1, wherein the composition comprises a liquid solution.

13. A method of treating skin, comprising topically applying the composition of the formulation of claim 1.

14. The method of claim 13, wherein the composition of the formulation is applied to the skin in the facial area.

15. The method of claim 14, wherein the composition of the formulation is applied to the skin using an oxygen machine.

16. The method of claim 14, wherein the composition of the formulation is applied to the skin using an air brush gun applicator.

17. A method of treating skin, comprising topically applying the composition of the formulation of claim 11.

18. The method of claim 17, wherein the composition of the formulation is applied to the scalp.

19. The method of claim 18, wherein the composition of the formulation is applied to the scalp using an oxygen machine.

20. The method of claim 18, wherein the composition of the formulation is applied to the skin using an air brush gun applicator.
